(19) United States
(12) Patent Application Publication
(10) Pub. No.: US 2022/0068152 A1
(43) Pub. Date: Mar. 3, 2022

(54) SYSTEM, APPARATUS, METHOD FOR ASSESSMENT OF AN ATHLETE'S PERFORMANCE IN AN ATHLETIC SPORT

(71) Applicants: Clarence R. Johnson, Stephenson, VA (US); Robert Wesley Smith, Centreville, VA (US); Regis Bolden, II, Centreville, VA (US); Sandeep Mehta, White Marsh, MD (US); Zuwana Johnson, Stephenson, VA (US); Javarro Russell, Moseley, VA (US); David Michael Schor, Argyle, TX (US)

(72) Inventors: Clarence R. Johnson, Stephenson, VA (US); Robert Wesley Smith, Centreville, VA (US); Regis Bolden, II, Centreville, VA (US); Sandeep Mehta, White Marsh, MD (US); Zuwana Johnson, Stephenson, VA (US); Javarro Russell, Moseley, VA (US); David Michael Schor, Argyle, TX (US)

(21) Appl. No.: 17/445,903

(22) Filed: Aug. 25, 2021

Related U.S. Application Data
(60) Provisional application No. 63/070,015, filed on Aug. 25, 2020.

(51) Int. Cl. G09B 7/00; G06F 3/0483; G06F 3/0484; A63B 24/00

(52) U.S. Cl. CPC G09B 7/00; A63B 24/0062; G06F 3/0484; G06F 3/0483

(57) ABSTRACT

A system, apparatus, and method to provide a systematic assessment learning approach that is utilized through a computing device to determine its users sport IQ score for one or more sports is disclosed. The system includes a multi-dimensional, systematic learning assessment processor to correlate answered questions and compute the sport IQ-score. In some cases, the questions in the database may include videos of sport content and other associated sport content. The system presents a quantifiable metric of a user's performance potential in a sport and provides a learning program for developing the user's skills across multiple dimensions.

### CROSS-REFERENCE TO RELATED APPLICATION

This application claims the benefit of priority of U.S. provisional application No. 63/070,015 filed Aug. 25, 2020, the contents of which are herein incorporated by reference.

### BACKGROUND OF THE INVENTION

The present invention relates to athletics, and more particularly to assessment tools for measuring an athlete's overall aptitude for performance in a selected sport.

Athletes, coaches, trainers, and referees, are often frustrated trying to close the gap between an athlete's conceptual knowledge of their sport and their actual physical athletic performance. This frustration gap may become apparent through the athlete's lack of playing time, being assigned to wrong playing positions, showing poor playing technique, misunderstanding of team systems, and general sub optimized athletic performance.

Further, there is no true data to measure and to confirm the athlete's knowledge gap within a selected sport. And there is no systematic methodology to correlate the knowledge gap across multiple game play dimensions and scenarios.

Currently there are no means, methods, or systems to assess and determine an athlete's IQ-score for a given sport. Further, there does not exist a systematic knowledge-based assessment methodology for the same.

As can be seen, there is a need for improved systems, apparatus, and method to provide a systematic knowledge-based assessment methodology for assessing an athlete's overall potential for performance of a selected sport.

### SUMMARY OF THE INVENTION

In one aspect of the present invention, a system for performance assessment of a participant in a sport is disclosed. The system includes a server hosted by a sport participant assessment service. The server is in communication with a database containing a repository of questions for assessing a participant metric across multiple domains of performance specified for the sport. The server hosts a plurality of web pages to assess the participant metric across the multiple domains of performance.

A first of the plurality of web pages is configured to present a user interface for a participant interaction with a quiz containing a plurality of questions pertaining to a knowledge domain for the sport. The user interface is configured to receive a participant answer to each of the plurality of questions. An embedded learning assessment processor is configured to determine a G-IQ score for the participant according to a compilation of the participant metric across the multiple domains of performance. The G-IQ score includes a knowledge domain component based on the participant answers to the plurality of questions.

In some embodiments, a second of the plurality of web pages is configured to present a user interface for recording a metric of the participant performance of the sport. The embedded learning assessment processor is configured to determine the G-IQ score for the participant within a performance domain based on the metric of the participant performance of the sport.

In some embodiments, the participant performance of the sport includes a competitive performance event.

In some embodiments, a third of the plurality of web pages is configured to present a user interface for recording a metric of a participant skill of the sport. The embedded learning assessment processor is configured to determine the G-IQ score for the participant within a skill domain based on the metric of the participant skill.

In some embodiments, a knowledge level configuration module is configured to provide a user interface for designating one or more knowledge levels of the sport within the knowledge domain.

In some embodiments, the knowledge level configuration module includes a weighting designation tool operable to set a weighting of the one or more knowledge levels. In some embodiments, the G-IQ is determined based on the weighting of the one or more knowledge levels.

In some embodiments, the knowledge level configuration module also includes a threshold designation tool configured to designate a threshold for determining the one or more knowledge levels.

In some embodiments, a quiz configuration module is configured to provide a user interface for designating one or more focus areas for a selected quiz and a user interface for designating a topic for each of the one or more focus areas.

In yet other embodiments, a user interface for designating one or more of a high and a low threshold for the selected quiz.

These and other features, aspects and advantages of the present invention will become better understood with reference to the following drawings, description and claims.

### BRIEF DESCRIPTION OF THE DRAWINGS

FIG. 1 is a schematic view of a representative system architecture for the athletics IQ assessment tool.

FIG. 2 is a schematic view of inputs to the athletics IQ assessment tool.

FIG. 3 is a schematic view of the athletics IQ assessment tool processes.

FIG. 4A is a flow chart illustrating the athletics IQ assessment tool processes for a first dimension.

FIG. 4B is a continuation of the flowchart of FIG. 4A, showing a quiz module.

FIG. 5 is a flow chart illustrating a second dimension assessment for the athletics IQ assessment tool.

FIG. 6 is a flow chart illustrating a third dimension assessment for the athletics IQ assessment tool.

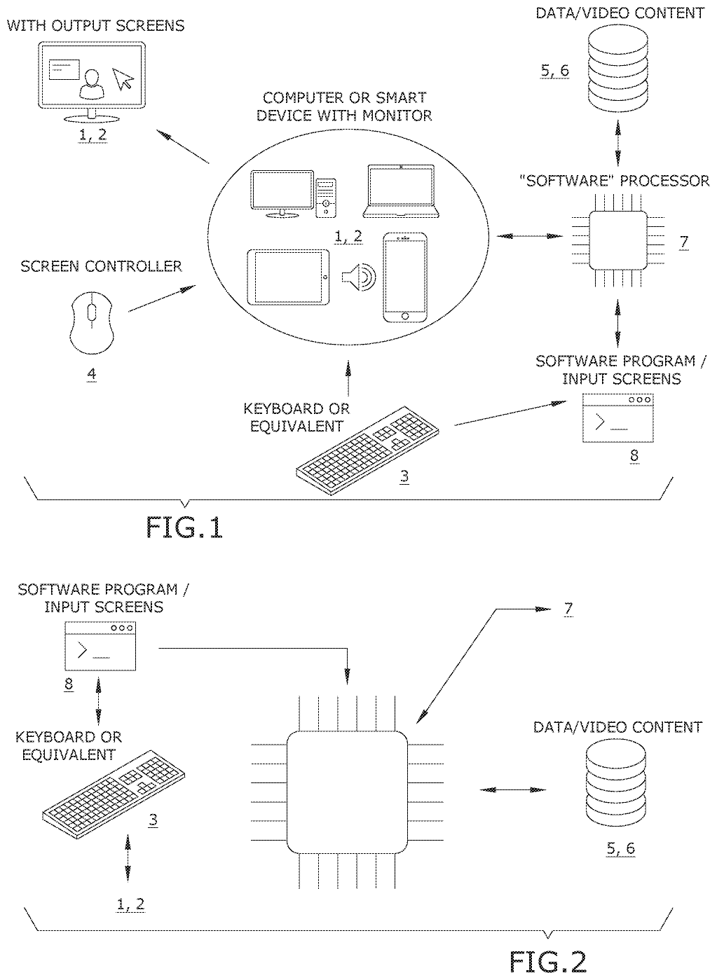

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, embodiments of the present invention provide a system, method, apparatus, and computer program product that provides for assessment of an athlete's performance in an athletic sport and produces their unique sport IQ-score. The user's IQ-score represents a user's measured understanding of the sport and may be used for individual, positional, team and competitive comparative purposes.

The system and software uses a multi-dimensional, systematic learning assessment processor to correlate answered questions and compute a sport IQ-score. In some cases, the questions in a database may include videos of sports content and other associated sport content. The user's sport IQ-score may then be used to identify knowledge gaps and allow for more focused training efforts for the athlete and team towards overall improved performance.

The present invention is an improvement on what currently exists. Currently there is no means to assess and determine an athlete's IQ-score for a given sport. Presently, users, including athletes, coaches, trainers, and referees, and the like, rely on eyesight, films, videos, playbooks, and scouting reports to make judgements on a player's skill level and potential playing performance. These inferior means do not establish an user's sport IQ-score or assess any knowledge gap the user may have to improve performance.

The software provides a systematic means to assess a user's knowledge of a sport and produce their unique sport IQ-score. The user's IQ-score represents a user's measured understanding of the sport and may be used for individual, positional, team and competitive comparative purposes.

Other systems lack a knowledge-based sport IQ scoring system and methodology. Without this capability, other devices or systems are limited and prevent athletes and teams from optimizing their full competitive potential.

As seen in reference to the drawings the system may include a computing device 1 and operating system 2 with an audio-input access, an audio-output capability, a visual input access, and a visual-output capability, commonly provided in a computer or a smart device. A display, such as a computer monitor or equivalent, a computer keyboard input device 3, mouse 4, or other human user interface device for controlling onscreen cursor activity with an input screen 8. A data base containing a data content 5 and a video content 6. A systematic learning assessment processor 7, for processing software programming language, data, and video input screens 8 and output screen 1, 2.

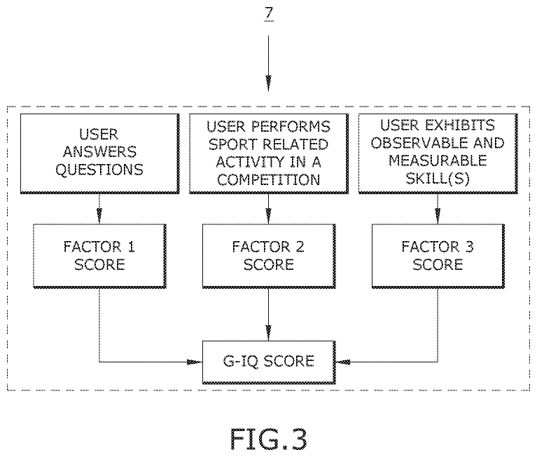

The system develops a sports IQ-score, hereinafter a G-Score, through a systematic learning assessment processor, such as shown in reference to FIG. 3. The system receives an input from a user's answers to content database questions and calculates the G-Score values across multiple dimensions. The system further correlates G-Score values across a learning scale of increasingly difficult question levels.

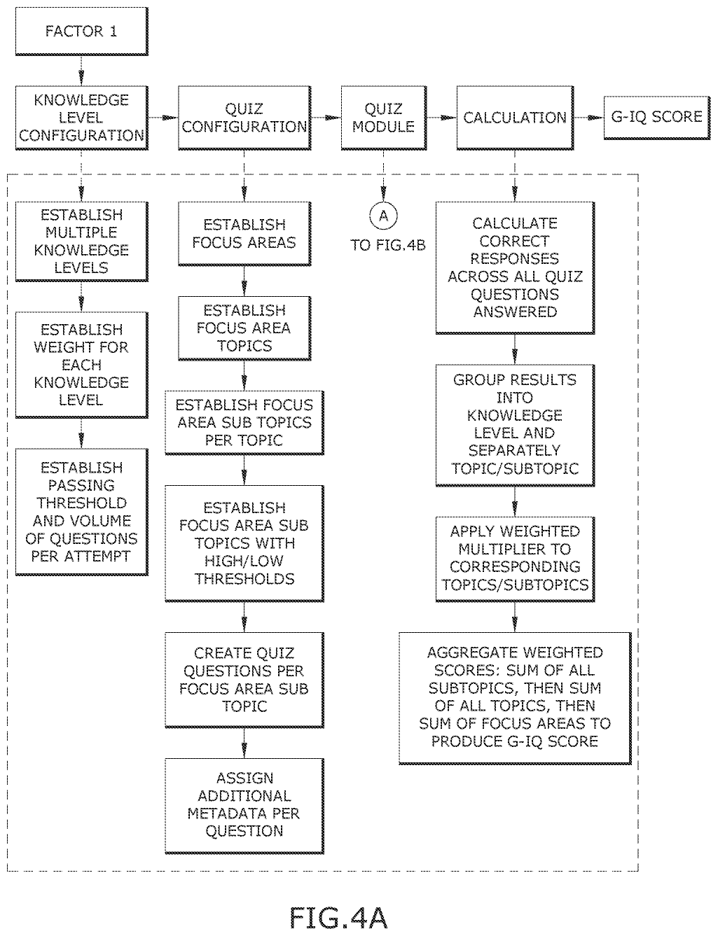

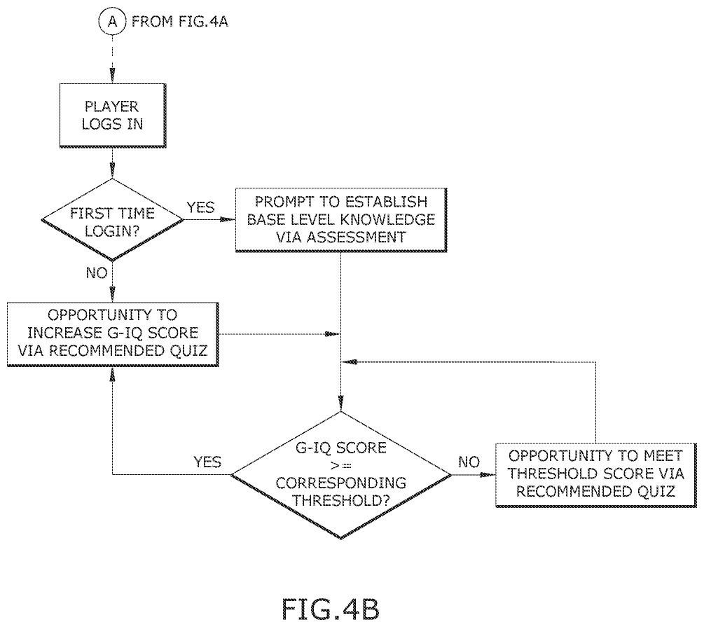

A representative assessment in a first dimension is shown in reference to FIG. 4A. A knowledge level configuration module for the first dimension is provided to allow for the establishment of multiple knowledge levels within the domain, such as novice, intermediate, advanced, master. The knowledge level may have weights established for the attainment of a particular knowledge level, a passing threshold for each knowledge level, and number of questions to be presented in an assessment session.

A quiz configuration module permits the specification of a focus area, establishment of one or more focus area topics, sub-topics for one or more topics. The quiz configuration module also permits for setting one or more thresholds for each topic/sub topic, which may be a high/low threshold. The quiz configuration module also provides for a quiz creation based on the foregoing parameters. The quiz configuration module may also provide for establishing additional meta data for each question. The meta data may be utilized for analytics purposes while the user takes a quiz, to identify performance parameters beyond the user getting the answer correct or incorrect during an assessment. The meta data may include capture of a dwell time on the question, user selection and changing of an answer, and other interactions with the question, when presented in the quiz.

The quiz module is shown in reference to FIG. 4B. After a user login the user is presented with an initial assessment quiz to establish a baseline knowledge assessment. After the quiz is completed, the user's G-IQ score is determined. If the user exceeds a predetermined threshold, the user is presented the opportunity to increase their G-IQ score with an additional recommended quiz, based on their current G-IQ score. If the user's G-IQ score does not exceed the predetermined threshold, the system offers the user the opportunity to meet the threshold by one or more recommended quizzes. The recommendations may be based on an assessment of the types of questions within the domain that the user has missed. For example, rules of the game versus tactics and strategies.

The calculation module determines the user's G-IQ score based on their interactions with the system. The calculation module first determines the number of correct responses across all quiz questions answered by the user. The results are grouped into a knowledge level across the one or more topics and subtopics. A weighted multiplier that may have been specified in the quiz configuration module based on an importance of the one or more topics or subtopics. The calculation module then aggregates the weighted scores. The sum of all the topics and a sum of one or more focus areas to provide the G-IQ score.

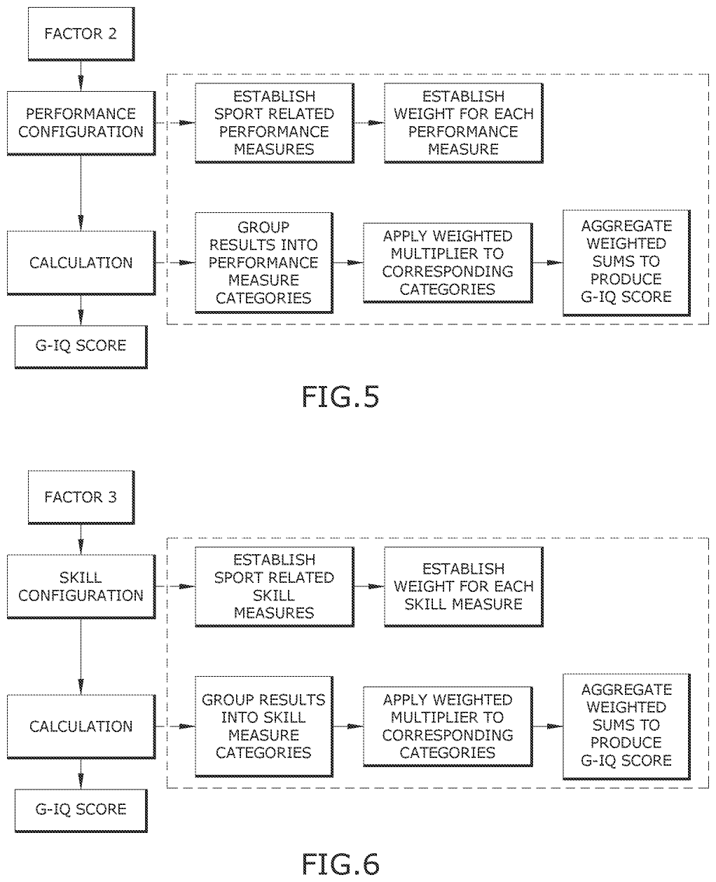

FIG. 5 illustrates an assessment for a second domain/factor, performance.

The second domain includes a performance configuration process, a calculation process, with an output of the G-IQ score. In the performance configuration process, the system includes establishment of performance measures for a particular sport. The performance configuration process also provides for a weighting of the one or more performance measures. The performance is typically assessed by the user's participation in the sports activity, more preferably in a competitive sports activity.

In the calculation process, the results are grouped into performance measure categories, the weighted multiplier, if any is applied, and the weighted sums are aggregated to produce the G-IQ score.

FIG. 6 illustrates an assessment for a third domain/factor, skill. The processes model those of the previous two domains, in this case, the assessments will be based on a skill relating to the particular sport. Unlike the performance domain, the skill domain may be assessed in observation or assessment of the user in the performance of particular skills or skill sets that the athlete may need perform to effectively participate in the sport.

The computer 1 acts as the central hub for this input and interaction activity, taking inputs from the users answers and allowing it to interact with the system software. The user perceives the content visually through the computer's video monitor 2. Through use of the keyboard 3 and mouse 4 or other similar human interface device, the user can proceed through the questions, answer the questions, and allow the system to derive their G-score. Results of the G-Score are displayed on one or more computer image screens 2. The displays 2 allow users to infer a relative comprehension (or deficiency) of subject matter questions from their G- Score.

An aspect of this system is its ability to determine level of knowledge for a user (i.e., athlete) using the system across multiple dimensions. The user will respond to several questions that will allow the software application to derive their G-Score. From there, the system will offer suggestions for further improvement of the user's knowledge and skills. The system provides a framework for developing sports knowledge provides users opportunities to achieve higher levels of success after developing and demonstrating foundational knowledge thresholds across multiple dimensions.

For each dimension of knowledge demonstrated, users may also earn badges, demonstrating their successful completion of a knowledge assessment. Once a dimension of the assessment is completed, the athlete will be provided opportunities to demonstrate their knowledge in other relevant areas of the game.

The system's overall logic framework is as follows:

```
    A broad score is computed per individually weighted
aggregation of information.
    If G-score < 1 {then do not display score}
    Else {execute the following function displayScore( )}
    Function displayScore( ) {
    For each subtopic: multiply a summation of all scores by a
pre-determined multiplier and corresponding difficulty level.
    For each topic: multiply the summation of all subtopic
scores by a pre-determined multiplier.
    For each focus area type: multiply the summation of all
topic scores by a pre-determined multiplier.
    For G-score: multiply the summation of all focus area
type scores by a pre-determined multiplier.}
```

Once the computer 1 set-up is complete and data content 5, 6 is perceptible on video monitor screen, user must login to the system and select “Players”. The Player selection will then advance to an “Assess Your Knowledge” view screen. The user then selects an assessment dimension to complete. The systematic learning assessment processor will then sequentially present database questions for user to answer. The user answers assessment questions, with the user's answers being stored. Once assessment is complete, a “Player Profile” screen may be presented. The Player Profile screen displays the user's assessment IQ-score (G-score).

The processor 7 will correlate G-score and display resulting dimensions for user's “Core Strength” and “Core Weakness”. The user may advance to another screen to graphically display the G-score as generated by the system. The system will also correlate the G-score and display user's “High Scores” across dimensions. The system may also display another screen to view user “Achievement Badges”.

In some aspects of the invention, the system may also provide another screen to allow the user to view “Recommendations to Increase Knowledge and Earn More Badges”. This recommendations screen allows for additional quiz-like questions for user to improve their G-score. The entire process can be repeated by the user for another assessment dimension from the “Assess Your Knowledge” view screen.

Additional, once computer 1 set-up is complete and data content 5, 6 is perceptible on video monitor screen 2, user can login to software and select “Coach”. The Coach selection will then advance to “Coach Profile” screen to view content. The user can also advance to another screen to view “^{˜}G-score Breakdown” for an entire team roster. The system correlates roster G-scores and displays results graphically across multiple dimensions.

The user may also advance to another screen to graphically display team's individual “User Engagement”. The system also links to another screen to view overall roster “Recommendations to Increase Knowledge and Earn More Badges”. This recommendations screen allows for additional quiz-like questions for team positions to improve G-score. This entire process can be repeated by the user for another assessment dimension from “Assess Your Knowledge” view screen.

The system of the present invention may include at least one computer with a user interface. The computer may include any computer including, but not limited to, a desktop, laptop, and smart device, such as, a tablet and smart phone. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

The computer-based data processing system and method described above is for purposes of example only, and may be implemented in any type of computer system or programming or processing environment, or in a computer program, alone or in conjunction with hardware. The present invention may also be implemented in software stored on a non-transitory computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A system for performance assessment of a participant in a sport, comprising:
a server hosted by a sport participant assessment service, the server in communication with a database containing a repository of questions for assessing a participant metric across multiple domains of performance specified for the sport, the server hosting a plurality of web pages to assess the participant metric across the multiple domains of performance;
a first of the plurality of web pages configured to present a user interface for a participant interaction with a quiz containing a plurality of questions pertaining to a knowledge domain for the sport, the user interface configured to receive a participant answer to each of the plurality of questions; and
an embedded learning assessment processor configured to determine a G-IQ score for the participant according to a compilation of the participant metric across the multiple domains of performance, wherein the G-IQ score includes a knowledge domain component based on the participant answers to the plurality of questions.

2. The system of claim 1, further comprising:
a second of the plurality of web pages configured to present a user interface for recording a metric of the participant performance of the sport; and
the embedded learning assessment processor configured to determine the G-IQ score for the participant within a performance domain based on the metric of the participant performance of the sport.

3. The system of claim 2, wherein the participant performance of the sport includes a competitive performance event.

4. The system of claim 1, further comprising:
a third of the plurality of web pages configured to present a user interface for recording a metric of a participant skill of the sport; and
the embedded learning assessment processor configured to determine the G-IQ score for the participant within a skill domain based on the metric of the participant skill.

5. The system of claim 1, further comprising:
a knowledge level configuration module configured to provide a user interface for designating one or more knowledge levels of the sport within the knowledge domain.

6. The system of claim 5, the knowledge level configuration module further comprising:
a weighting designation tool operable to set a weighting of the one or more knowledge levels.

7. The system of claim 6, wherein the G-IQ is determined based on the weighting of the one or more knowledge levels.

8. The system of claim 6, the knowledge level configuration module further comprising:
a threshold designation tool configured to designate a threshold for determining the one or more knowledge levels.

9. The system of claim 8, further comprising:
a quiz configuration module configured to provide a user interface for designating one or more focus areas for a selected quiz; and
a user interface for designating a topic for each of the one or more focus areas.

10. The system of claim 9, further comprising:
a user interface for designating one or more of a high and a low threshold for the selected quiz.
